(19) United States
(12) Patent Application Publication
(10) Pub. No.: US 2006/0039874 A1
(43) Pub. Date: Feb. 23, 2006

(54) Teeth whitening device

(76) Inventor: Hammond Wong, West Covina, CA (US)

Correspondence Address: Mr. Walter J. Tencza Jr., Suite 3, 10 Station Place, Metuchen, NJ, 08840, US

(21) Appl. No.: 11/049,254

(22) Filed: Feb. 2, 2005

Related U.S. Application Data
(63) Continuation-in-part of application No. 10/924,062, filed on Aug. 23, 2004.

(51) Int. Cl. A61K 8/22; A61C 5/00

(52) U.S. Cl. 424/49; 433/217.1

(57) ABSTRACT

A device is provided which includes a cavity containing teeth whitening gel. The device can be attached to a tooth so that the tooth lies inside the cavity and the teeth whitening gel comes in contact with the tooth. The device may includes a plurality of sections, each of which may be designed to attach onto a corresponding single tooth. Each of the plurality of sections may have a width and a length which are about the same as the width and the length, respectively, of its corresponding single tooth. The device can be compressed, expanded, or flexed, so that the device can fit different sets of teeth for different individuals. The apparatus may further include a protective covering for sealing the gel inside the cavity.

CROSS REFERENCE TO RELATED APPLICATION(S)

This application is a continuation in part of and claims the priority of U.S. patent application Ser. No. 10/924,062, filed on Aug. 23, 2004, titled “TEETH WHITENING DEVICE”, inventor and applicant Hammond Wong.

### FIELD OF THE INVENTION

This invention relates to improved methods and apparatus concerning whitening or cleaning teeth.

### BACKGROUND OF THE INVENTION

Various methods and devices are known in the prior art which are used for whitening teeth.

### SUMMARY OF THE INVENTION

The present invention, in one or more embodiments, provides an apparatus comprising a device which includes a cavity. The cavity typically is filled with teeth whitening gel or another teeth health chemical or combination of teeth health chemicals. The device can be attached to a tooth so that the tooth lies inside the cavity and the teeth whitening gel comes in contact with the tooth.

The device may include a plurality of sections. Each of the plurality of sections may be designed to attach onto a corresponding single tooth. Each of the plurality of sections may have a width and a length which are about the same as the width and the length, respectively, of its corresponding single tooth.

The device can be compressed or expanded so that the device can fit different sets of teeth for different individuals. The device can be flexed to allow for adjustment for different teeth configurations for different individuals. The device may be in the form of a flexible, expandable, and compressible straw or may be made of elastic materials.

The apparatus may further include a protective covering. The protective covering may be attached to the device and may keep the teeth whitening gel sealed inside the device until the protective covering is at least partially removed from the device.

The present invention also provides a method comprising the steps of providing a device which includes a cavity and inserting teeth whitening get into the cavity. The device can be attached to a tooth so that the tooth lies inside the cavity and the teeth whitening gel comes in contact with the tooth.

### BRIEF DESCRIPTION OF THE DRAWINGS

FIG. 1A shows a perspective view of an apparatus comprised of upper and lower (or first and second) teeth whitening devices attached to a holder;

FIG. 1B shows a perspective view of the upper and lower teeth whitening devices of FIG. 1A detached from the holder of FIG. 1A;

FIG. 2A shows a perspective view of the upper teeth whitening device of FIG. 1A with a protective cover attached to the upper teeth whitening device so that gel is sealed within the combination of the upper teeth whitening device and the protective cover;

FIG. 2B shows a perspective view of the combination of the upper teeth whitening device and the protective cover, with the protective cover partially removed from the upper teeth whitening device;

FIG. 2C shows a perspective view of the upper teeth whitening device without the protective cover;

FIG. 3A shows a perspective view of the upper teeth whitening in an at rest state where the upper teeth whitening device is neither being compressed or stretched;

FIG. 3B shows a perspective view of the upper teeth whitening in a stretched or expanded state where the upper teeth whitening device is being stretched;

FIG. 4A shows a cross sectional view of the upper teeth whitening device and part of the holder of FIG. 1A;

FIG. 4B shows a cross sectional view of the upper teeth whitening device, including gel within the upper teeth whitening device;

FIG. 4C shows a cross sectional view of the upper teeth whitening device attached to one or more teeth of a human being;

FIG. 4D shows a side view of the protective cover and the upper teeth whitening device;

FIG. 4E shows a cross sectional view of the protective cover partially removed from the upper teeth whitening device;

FIG. 5A shows a perspective view of another upper teeth whitening device being held by a person;

FIG. 5B shows a perspective view of the upper teeth whitening device of FIG. 5A attached to teeth of a person;

FIG. 6A shows a perspective view of an upper teeth whitening device in an at rest state, having stretchable sections and a non-stretchable section;

FIG. 6B shows a perspective view of the upper teeth whitening device of FIG. 6A in a stretched state;

FIG. 7A shows a perspective view of the upper teeth whitening device of FIG. 6A being held by a person;

FIG. 7B shows a perspective view of the upper teeth whitening device of FIG. 6A attached to teeth of a person;

FIG. 8A shows a perspective view of an apparatus in accordance with another embodiment of the present invention;

FIG. 8B shows a transparent view of the apparatus if FIG. 8A;

FIG. 9A shows a cross sectional view of a portion of the apparatus of FIG. 8A along with gel inserted into the portion of the apparatus;

FIG. 9B shows a cross sectional view of the portion of FIG. 9A along with gel and a tooth inserted into the portion of the apparatus; and

FIG. 9C shows a cross sectional view of the portion of FIG. 9A along with gel and a tooth inserted into the portion of the apparatus and with pressure applied to an outside portion of the apparatus.

### DETAILED DESCRIPTION OF THE DRAWINGS

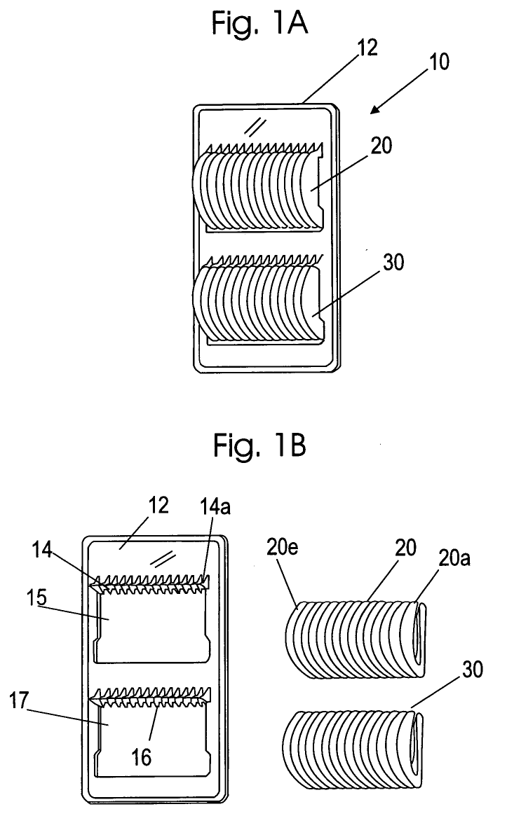

FIG. 1A shows a perspective view of an apparatus 10 comprised of upper and lower (or first and second) teeth whitening devices 20 and 30 attached to a holder 12. FIG. 1B shows a perspective view of the upper and lower teeth whitening devices 20 and 30, respectively, of FIG. 1A detached from the holder of FIG. 1A.

As shown in FIG. 1B, the holder 12 includes protrusions or “fake” teeth 14 and 16 to which the whitening devices 20 and 30, respectively, can be attached. The holder 12 also has openings 15 and 17 into which whitening devices 20 and 30, respectively, can be inserted.

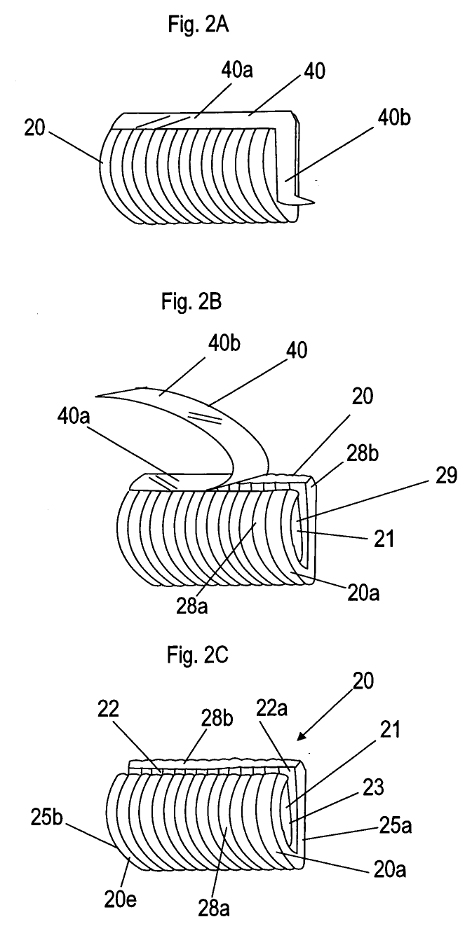

FIG. 2A shows a perspective view of the upper teeth whitening device 20 of FIG. 1A with a protective cover 40 attached to the upper teeth whitening device 20 so that gel 21 (whose location is shown in FIG. 2B) is sealed within the combination of the upper teeth whitening device 20 and the protective cover 40. FIG. 2B shows a perspective view of the combination of the upper teeth whitening device 20 and the protective cover 40, with the protective cover 40 partially removed from the upper teeth whitening device 20. FIG. 2C shows a perspective view of the upper teeth whitening device 20 without the protective cover 40. The protective cover 40 may include a first section 40a which is designed to fit over opening or channel 22 (shown in FIG. 2C) of the device 20 and a second section 40b which is designed to cover side opening 29 shown in FIG. 2B.

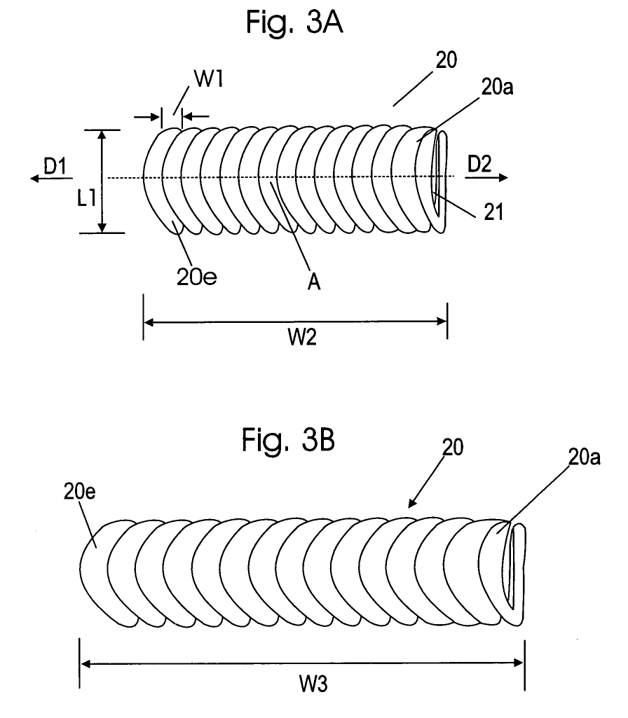

The whitening devices 20 and 30 may be identical. The whitening device 20 will be described in further detail. As shown in FIGS. 2C, 3A, and 3B the whitening device 20 may include a plurality of sections such as sections 20a and 20e. Each section, such as section 20a or section 20e, is designed to enclose and attach to a human tooth. Each section, such as section 20a or section 20e, has a width, such as W1 and a length such as L1 shown in FIG. 3A. The width and length of each section, such as section 20a or 20e, may correspond to the width and length, respectively of a specific tooth, for example of a left front tooth of an average adult male human being. Each section, of upper teeth whitening device 20 may also be designed to attach to a specific protrusion of protrusions 14. For example section 20a shown in FIG. 3A may be designed to attach to protrusion 14a of the holder 12 shown in FIG. 1B.

Each section, such as 20a or 20e, can be u-shaped as shown in FIG. 2C, and each section may have a top opening, such as top opening 22a for section 20a. All of the top openings of the sections 20 form top opening or channel 22 as shown in FIG. 2C. A plurality of teeth can be inserted into the top opening 22. The upper teeth whitening device 20 may have end walls 25a and 25b within which may reside a cavity, indentation or depression 23. Gel 21 can reside within the cavity 23, typically near the bottom of the u-shaped sections (such as section 20a) of device 20, within the walls 25a and 25b as shown in FIG. 2C.

FIG. 3A shows a perspective view of the upper teeth whitening device 20 in an at rest state where the upper teeth whitening device 20 is neither being compressed or stretched. In the at rest state the whitening device 20 has a width of W2 inches, which may be one inch. FIG. 3B shows a perspective view of the upper teeth whitening device 20 in a stretched or expanded state. In a stretched state, the whitening device 20 may have a width of W3 which may be two inches.

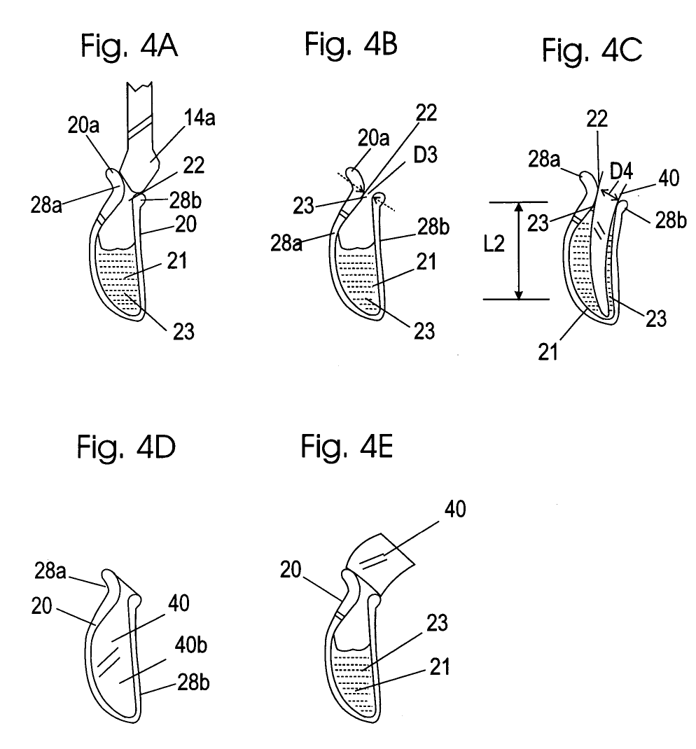

FIG. 4A shows a cross sectional view of the upper teeth whitening device 20 and part or protrusion 14a of the holder 14 of FIG. 1A. FIG. 4B shows a cross sectional view of the upper teeth whitening device 20 including gel 21 within the upper teeth whitening device 20. FIGS. 4A and 4B also show front portion 28a and rear portion 28b of the device 20, top opening or channel 22 of the device 20, and gel 21 within cavity 23 of the device 20. As shown in FIG. 4B, the top opening or channel 22 would be D3 inches wide when front and rear portions 28a and 28b are not being forced apart or pushed together, i.e. are at rest with respect to each other.

FIG. 4C shows a cross sectional view of the upper teeth whitening device 20 attached to one or more teeth of a human being, including a tooth 40. In FIG. 4C, the tooth 40 has a length of L2, which approximately corresponds to the length L1 of the cavity 23 of the whitening device 20. As shown in FIG. 4C the top opening or channel 22 is now D4 inches, which is greater than D3 in FIG. 4B. In the state of FIG. 4C, the front and rear portions 28a and 28b have been forced apart from one another. The front and rear portions 28a and 28b frictionally hold onto the tooth 40 and to other teeth not shown.

Note that the walls 25a and 25b, shown in FIG. 2C, are not shown in FIGS. 4A, 4B, and 4C, but would typically be present to keep gel 21 from falling out of the cavity 23 of the whitening device 20. The wall 25a may be higher than shown in FIG. 2C.

FIG. 4D shows a side view of the protective cover 40 and section 40b attached to the whitening device 20. FIG. 4E shows a cross sectional view of the protective cover 40 partially removed from the upper teeth whitening device 20. Note again that the walls 25a and 25b are not shown in FIG. 4E, but would typically be present to keep gel 21 from falling out of the cavity 23 of the whitening device 20.

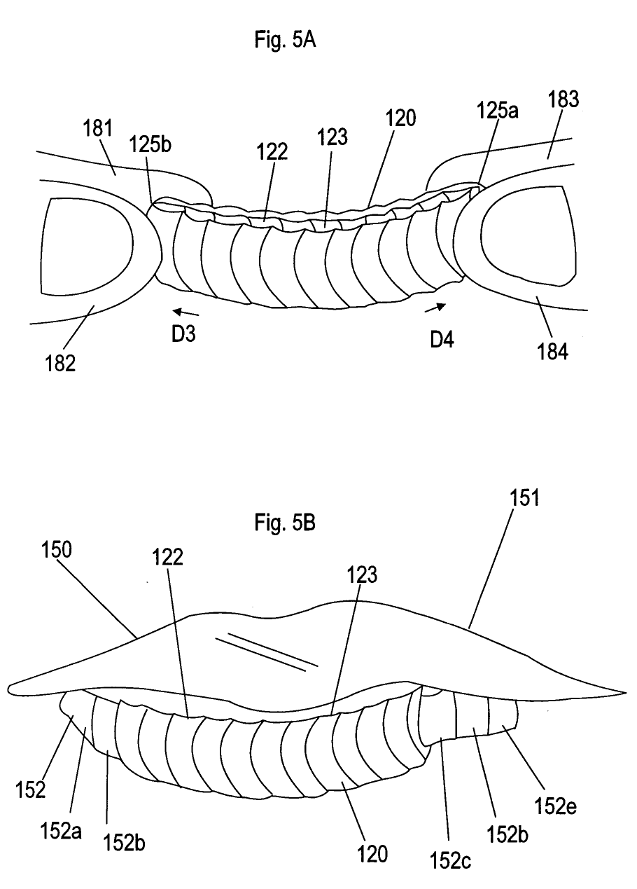

FIG. 5A shows a perspective view of another upper teeth whitening device 120 being held by a person having left hand fingers 181 and 182 and right hand fingers 183 and 184. The whitening device 120 has end or end walls 125a and 125b. The fingers 183 and 184 hold end 125a and the fingers 181 and 182 simultaneously hold end 125b. End 125a is pulled in a direction D4 while the end 125b is simultaneously pulled in a direction D3 in order to stretch the upper teeth-whitening device 120. FIG. 5B shows a perspective view of the upper teeth whitening device 120 of FIG. 5A attached to teeth 152 of a person. Lips 150 of the person's mouth 151 are also shown. The teeth 152 include teeth 152a-e and further teeth which are not shown and which are covered and lie within the cavity 123 of the teeth-whitening device 120. The teeth whitening device 120 may be similar to or identical to the teeth whitening device 20 of FIG. 1A. FIGS. 5A and 5B also show the location of opening or channel 122.

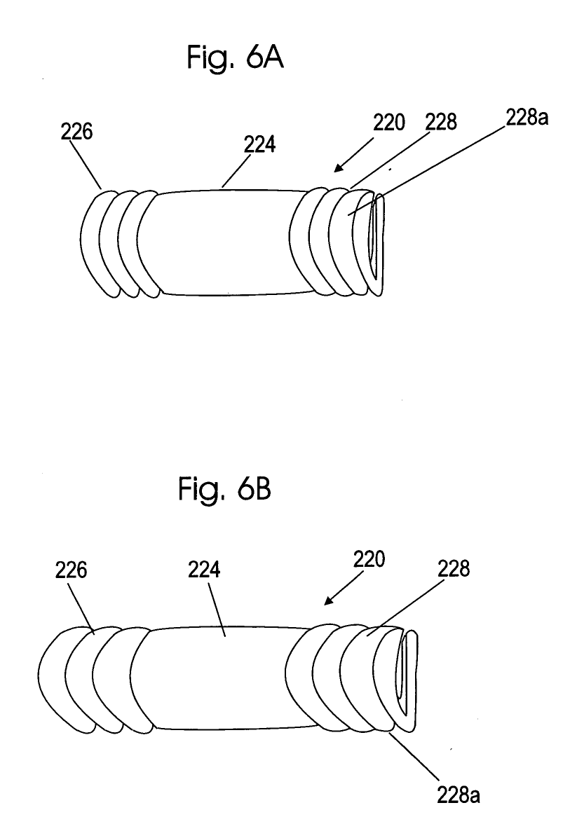

FIG. 6A shows a perspective view of an upper teeth whitening device 220 in a rest state, having stretchable sections 226 and 228 and a non-stretchable center section 224. The whitening device 220 may be similar to the whitening device 20 and 120, except for the non-stretchable center section 224. The sections 228 may include section 228a. The sections 226 may include a plurality of sections similar to section 228a. FIG. 6B shows a perspective view of the upper teeth whitening device 220 of FIG. 6A in a stretched state. Sections 226 and 228 are shown stretched in FIG. 6B while section 224 is not stretched.

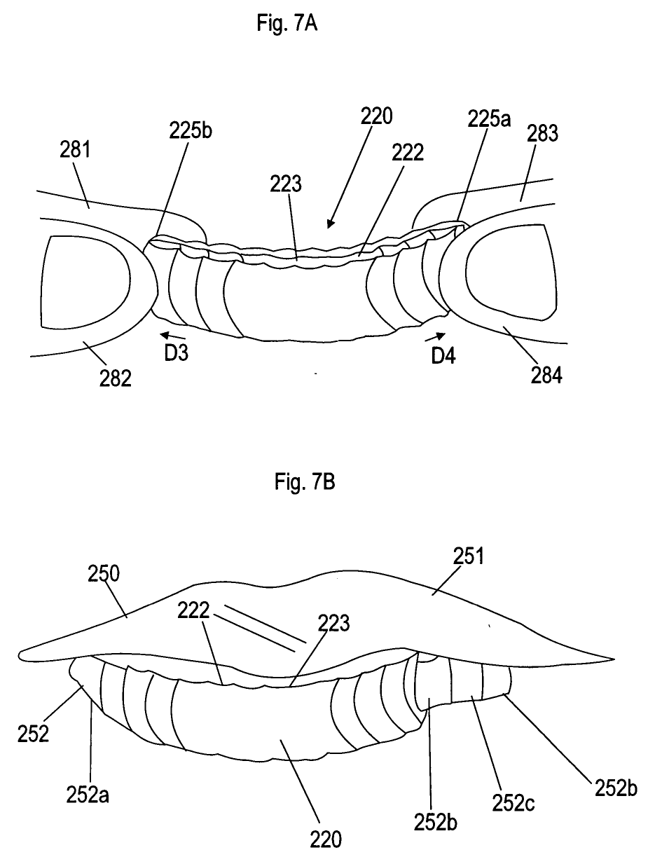

FIG. 7A shows a perspective view of the upper teeth whitening device 220 of FIG. 6A being held by a person. Fingers 281 and 282 are shown holding end 225b and fingers 283 and 284 are shown holding end 225a. End 225a is pulled in the direction D4 while end 225b is simultaneously pulled in a direction D3 in order to stretch the upper teeth-whitening device 220. FIG. 7B shows a perspective view of the upper teeth whitening device 220 of FIG. 7A attached to teeth 252 of a person. Lips 250 of the person's mouth 251 are also shown. The teeth 252 include teeth 252a-d and further teeth which are not shown and which are covered and lie within the cavity 223 of the teeth-whitening device 220. The teeth whitening device 220 may be similar to or identical to the teeth whitening device 20 of FIG. 1A or 120 of FIG. 5A. FIGS. 7A and 7B also show the location of opening or channel 222.

The whitening devices 20 can be bent, so that the axis A of the whitening device 20 shown in FIG. 3A is bent. The whitening device 20 can be compressed by pushing inwards on the end wall 25a and simultaneously pushing inwards on the end wall 25b shown in FIG. 2C. The ability to bend, expand, and compress the whitening device 20 allows the device 20 to attach to different size teeth of different individuals. The whitening device 20, with the exception of the gel 21, may be an expandable, bendable, and compressible straw, such as a plastic straw.

FIG. 1A along with FIG. 1B shows one way of packing the whitening devices 20 and 30. FIGS. 2A, 2B, and 2C show a second way of packing the device 20. The device 30 can also be packed in the manner of FIGS. 2A-C. Each section, such as section 20a, is typically attached by a thin member of the same material to its respective protrusion such as protrusion 14a. To detach device 20, one needs to simply push 20 and the material connecting 14a and 20a, and other sections to other protrusions, will break apart and allow device 20 to release from teeth 14.

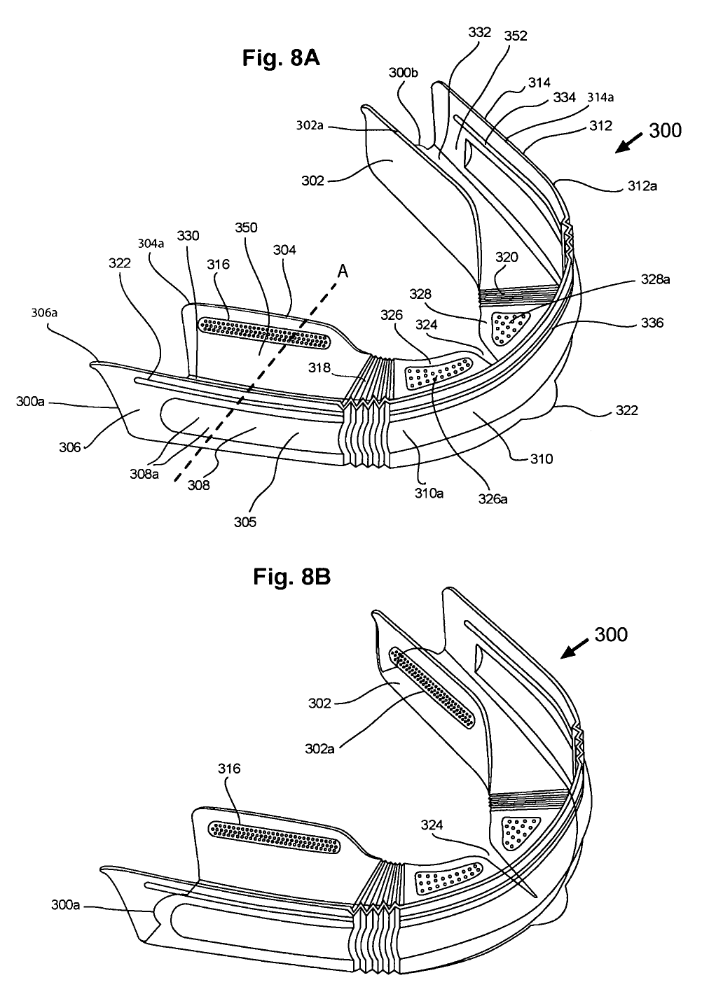

FIG. 8A shows a perspective view of an apparatus 300 accordance with another embodiment of the present invention. The apparatus 300 includes inner walls 302 and 304, outer wall 306, curved outer wall portions 308, 310, and 312, outer wall 314, section 316, pleated or accordion sections 318 and 320, center grip 322, center gap or slot 324, and bases or bottom portions 326, 328, 330, and 332. The base or bottom portion 330 is connected to the base or bottom portion 326 through pleated section 318. The wall 306 is connected to the wall 310 by pleated section 318. The wall 304 is connected to the base 326 by the pleated section 318. The base 332 is connected to the base 328 by pleated section 320. The wall 314 is connected to the wall 310 by pleated section 320. The wall 302 is connected to the base 328 by pleated section 320. The outer wall 306 has an end 306a which projects outward and the inner wall 304 has an end 304a which projects outward. The projecting outwards of ends 306a and 304a makes it easier to insert a tooth, such as tooth 402. The outer wall 314 and inner wall 302, similarly have ends 314a and 302a, respectively, which project outwards. The projecting outwards of ends 314a and 302a makes it easier to insert a tooth, such as tooth 402.

The pleated sections 318 and 320 can expand, contract, or bend. The purpose of the pleated sections 318 and 320 is to allow the apparatus 300 to fit different teeth sizes and shapes for different human beings. The section 316 on the inside of wall 304 provides a rough ridge which attached to the back of teeth in a human being's mouth. The roughness creates friction in between the apparatus or tray 300 and the teeth. The purpose of the ridge or section 316 is to prevent the apparatus or tray 300 from falling off of the teeth. There may be a similar ridge or section provide on the inside of wall 302. The section, portion or curved walls 308, 310, and 312 form a gel trench, which is used to hold teeth whitening gel. Gel is released from the inside walls 308, 310, and 312 by pressing on the outer surfaces 308a, 310a, and 312a.

The apparatus or tray 300 may have ends or end grips 300a and 300b. The apparatus or tray 300 can be expanded, contracted or bent by gripping the center grip 322 and the end grip 300a or the end or end grip 300b simultaneously or the apparatus or tray 300 can also be expanded, contracted or bent by gripping the end grip 300a and the end or end grip 300b simultaneously in order to conform the apparatus or tray 300 to a particular person's set of teeth in a person's mouth or in order to remove the apparatus or tray 300 from a set of teeth in a person's mouth.

FIG. 8B shows a transparent view of the apparatus 300. The transparent view shows ridges 302a or ridge section 302a which are similar to ridges 316 and are used for frictionally engaging teeth. The ridges 302a are on the inside of wall 302. FIG. 8B also shows the location of end or end grip 300a.

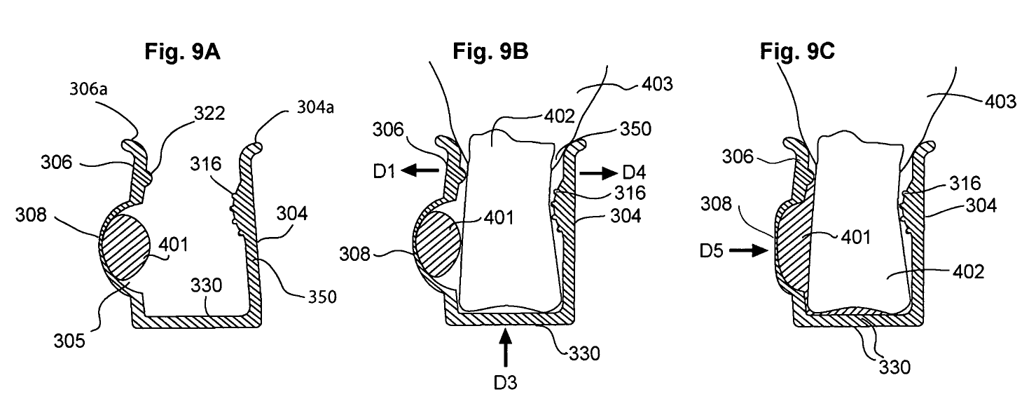

FIG. 9A shows a cross sectional view of a portion of the apparatus 300 of FIG. 8A along dashed line A show in FIG. 8A. FIG. 9A shows inner wall 304, outer walls 306 and 308 and base 330. FIG. 9A also shows ridge 316 and ridge or protrusion 322. FIG. 9A also shows a cross sectional view of the portion of gel 401 inserted into the portion of the apparatus 300. The gel 401 can be injected in the indentation or cavity 305 either manually by using a syringe or automatically by means of automation. FIG. 9B shows a cross sectional view of the portion of FIG. 9A of apparatus 300 along with gel 401 and a tooth 402 inserted into the portion of the apparatus 300. The tooth 402 is attached to gums 403. As shown by FIG. 9B, the ridge 322 frictionally engages an outer portion of tooth 402 while ridge section 316 frictionally engages an inner surface of tooth 402. The ridge 322 and corresponding ridges 336 and 334, also act as a barrier to prevent the gel 401 from leaking into the gingival area, i.e. near gums 403 or the area where the tooth 402 meets the gums 403, which is sensitive to the gums.

FIG. 9B shows that the wall 306 can be forced or expanded outward in the direction of D1 while the wall 304 is forced outward in the direction of D4, to remove the apparatus 300 from the set of teeth including tooth 401. FIG. 9B also shows that the bottom portion or base 330 can be pushed upwards in the direction of D3 to insert the tooth 402 (and other teeth not show) into the opening, gap or cavity 350 of the apparatus 300. The opening, gap or cavity 350 is defined by outer wall 306, based 330, and inner wall 304. Another similar or identical opening gap or cavity 352 is defined by outer wall 314, base 332 and inner wall 302. There is an indentation 305 in the curved portion 308 of outer wall 306 in which gel 401 is located as shown in FIG. 9A.

In FIG. 9B there is no outer pressure applied to wall 308. In FIG. 9C, pressure in a direction of D5 can be applied by pressing with a finger on the outer wall 308. This pressing causes the gel 401 to be forced to spread more evenly around the tooth 402, such as over the outer wall 402a, on the bottom 402a and near the top 402c of tooth 402, and spread evenly in a similar manner over other teeth.

The entire apparatus 300 may be made of ethylene vinyl accetate (EVA) or polyethylene terephthalate (PETE). The gap 324 is expandable so as to accommodate different shapes or sizes of teeth. The walls 302 and 314 and the bottom or base 332 are flexible to make insertion easier. Similarly the walls 304, 306 and the bottom or base 330 are flexible to make insertion easier. The bottom or bases 326 and 328 include frictional portions or ridges 326a and 328a which frictionally engage the inner walls and bottoms of a tooth or teeth to hold the apparatus 300 stationary with respect to the tooth or teeth.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

I claim:

1. An apparatus comprising:
A first outer wall;
A first inner wall;
A first base;
wherein the first outer wall, the first inner wall, and the first base define a first cavity;
wherein the first cavity includes first teeth whitening gel; and
wherein the first outer wall, first inner wall and first base can be attached to a first tooth so that the first tooth lies inside the first cavity and the first teeth whitening gel comes in contact with the first tooth;
and wherein the apparatus includes a first inner ridge for frictionally engaging at least one tooth:

2. The apparatus of claim 1 wherein
the first inner ridge is connected to the first inner wall.

3. The apparatus of claim 1 wherein
the first inner ridge is connected to the first outer wall.

4. The apparatus of claim 1 wherein
the first inner ridge is connected to the first base.

5. The apparatus of claim 1 wherein
the first gel is located in the first cavity adjacent the first outer wall; and
wherein the first gel can be released by pressing on a first outer surface of the first outer wall.

6. The apparatus of claim 1 further comprising
a first pleated section which allows the apparatus to expand, contract, and bend.

7. The apparatus of claim 6 wherein
the first pleated section has a first portion which is part of the first outer wall;
and the first pleated section has a second portion which is part of the first base.

8. The apparatus of claim 6 further comprising
a second outer wall;
a second inner wall;
a second base;
wherein the second outer wall, the second inner wall, and the second base define a second cavity;
wherein the second cavity includes second teeth whitening gel; and
wherein the second outer wall, the second inner wall and second base can be attached to a second tooth so that the second tooth lies inside the second cavity and the second teeth whitening gel comes in contact with the second tooth;
wherein the apparatus includes a second inner ridge for frictionally engaging at least one tooth; and
further comprising a second pleated section which allows the apparatus to expand, contract, and bend.

9. The apparatus of claim 8 wherein
the first pleated section has a first portion which is part of the first outer wall;
the first pleated section has a second portion which is part of the first base;
the second pleated section has a first portion which is part of the second outer wall; and
and the second pleated section has a second portion which is part of the second base.

10. A method comprising the steps of:
providing a device which includes a first cavity;
inserting first teeth whitening gel into the first cavity;
wherein the device can be attached to a first tooth so that a first tooth lies inside the first cavity and the first teeth whitening gel comes in contact with the first tooth;
and wherein the device includes a first inner ridge for frictionally engaging the back of at least one tooth.

11. The method of claim 10 wherein
the device includes a first outer wall, a first inner wall, and a first base;
wherein the first outer wall, the first inner wall, and the first base define the first cavity;
wherein the first outer wall, the first inner wall and the first base can be attached to a first tooth so that the first tooth lies inside the first cavity and the first teeth whitening gel comes in contact with the first tooth; and
wherein the first inner ridge is connected to the first inner wall.

12. The method of claim 10 wherein
the device includes a first outer wall, a first inner wall, and first base;
wherein the first outer wall, first inner wall, and first base define the first cavity;
wherein the first outer wall, first inner wall and first base can be attached to a first tooth so that the first tooth lies inside the first cavity and the first teeth whitening gel comes in contact with the first tooth; and
the first inner ridge is connected to the first outer wall.

13. The method of claim 10 wherein
the device includes a first outer wall, a first inner wall, and first base;
wherein the first outer wall, the first inner wall, and the first base define the first cavity;
wherein the first outer wall, the first inner wall and the first base can be attached to a first tooth so that the first tooth lies inside the first cavity and the first teeth whitening gel comes in contact with the first tooth; and
the first inner ridge is connected to the first base.

14. The method of claim 10 wherein
the device includes a first outer wall, a first inner wall, and a first base;
wherein the first outer wall, the first inner wall, and the first base define the first cavity;
the first gel is located in the first cavity adjacent the first outer wall; and
wherein the first gel can be released by pressing on a first outer surface of the first outer wall.

15. The method of claim 10 wherein
the device includes a first pleated section which allows the device to expand, contract, and bend.

16. The method of claim 11 wherein
the first pleated section has a first portion which is part of the first outer wall;
and the first pleated section has a second portion which is part of the first base.

17. The method of claim 15 wherein
the device includes
a second outer wall;
a second inner wall;
a second base;
wherein the second outer wall, the second inner wall, and the second base define a second cavity;
wherein the second cavity includes second teeth whitening gel; and
wherein the second outer wall, the second inner wall and the second base can be attached to a second tooth so that the second tooth lies inside the second cavity and the second teeth whitening gel comes in contact with the second tooth;
wherein the device includes a second inner ridge for frictionally engaging at least one tooth; and
wherein the device further includes a second pleated section which allows the device to expand, contract, and bend.

18. The method of claim 17 wherein
the first pleated section has a first portion which is part of the first outer wall;
the first pleated section has a second portion which is part of the first base;
the second pleated section has a first portion which is part of the second outer wall; and
and the second pleated section has a second portion which is part of the second base.
